(19) United States
(12) Patent Application Publication
(10) Pub. No.: US 2004/0009932 A1
(43) Pub. Date: Jan. 15, 2004

(54) Antibacterial compounds with activity against penicillin-resistant streptococcus pneumoniae

(76) Inventors: Kathleen Phelan, Gurnee, IL (US); Stevan Djuric, Libertyville, IL (US); Zhenkun Ma, Dallas, TX (US); Thomas Marron, Libertyville, IL (US); Hong Yong, Grayslake, IL (US); Irini Zanze, Lake Bluff, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/421,460

(22) Filed: Apr. 23, 2003

Related U.S. Application Data
(60) Provisional application No. 60/375,652, filed on Apr. 26, 2002.

(51) Int. Cl. C07H 17/08; A61K 31/7048

(52) U.S. Cl. 514/29; 536/7.4

(57) ABSTRACT

Compounds which are useful as antibacterials for penicillin-resistant streptococcus pneumoniae and having formula (I)

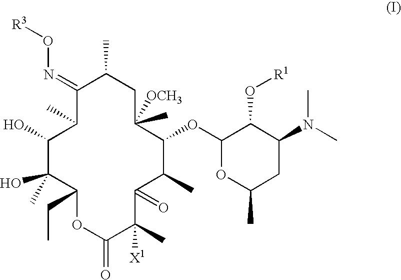

and formula (II)

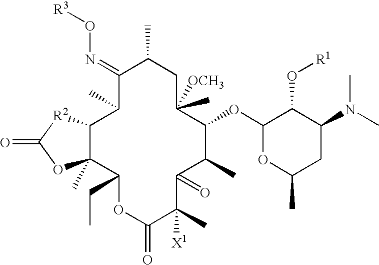

and salts, prodrugs, and salts of prodrugs thereof, processes for making the compounds and intermediates used in the processes, compositions containing the compounds, and methods for prophylaxis or treatment of bacterial infections using the compounds are disclosed.

This application claims benefit of co-pending U.S. Provisional Application Ser. No. 60/375,652, filed Apr. 26, 2002, the specification of which is hereby incorporated by reference into this application.

### TECHNICAL FIELD

This invention is directed to compounds which have activity against penicillin-resistant streptococcus pneumoniae, processes for making the compounds and intermediates used the processes, compositions containing the compounds, and methods for prophylaxis or treatment of penicillin-resistant streptococcus pneumoniae using the compounds.

### BACKGROUND OF THE INVENTION

Because the effectiveness of many drugs currently available for prophylaxis or treatment of penicillin-resistant streptococcus pneumoniae is being compromised by drug-resistance, novel compounds with activity against penicillin-resistant streptococcus pneumoniae would be beneficial for their therapeutic value and their contribution to the antibacterial arts.

### SUMMARY OF THE INVENTION

A first embodiment of this invention, therefore, is directed to compounds which are useful against penicillin-resistant streptococcus pneumoniae, and salts, prodrugs, and salts of prodrugs thereof, the compounds having formula (I)

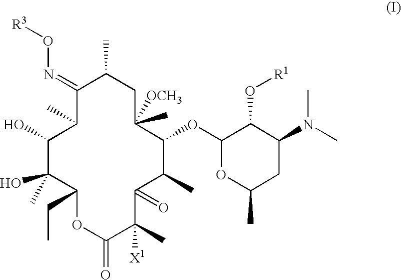

and formula (II)

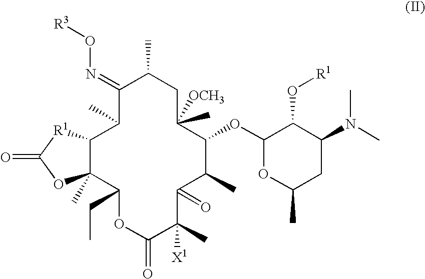

in which

R¹ is hydrogen or R^{P}, in which R^{P} is a hydroxyl protecting moiety;

R² is —O— or —NH—;

R³ is —CH₂R⁴, —CH₂CH₂R⁵, or —CH₂CH₂R⁶;

R⁴ is alkyl interrupted with one, two, three, or four moieties independently selected from the group consisting of —O—, ═N—, —NH—, —N(alkyl)-, —S—, —S(O)—, and —S(O)₂—, substituted with one substituent selected from the group consisting of —NH₂, —NHR⁷, and —NR⁷R⁸, and further unsubstituted or substituted with one or two ═O substituents;

R⁵ is alkenyl interrupted with one or two moieties independently selected from the group consisting of —O—, ═N—, —NH—, —N(alkyl)-, —S—, —S(O)—, and —S(O)₂—, substituted with one substituent selected from the group consisting of —NH₂, —NHR⁹, and —NR⁹R¹⁰, and further unsubstituted or substituted with one or two ═O substituents;

R⁶ is alkynyl interrupted with one or two moieties independently selected from the group consisting of —O—, ═N—, —NH—, —N(alkyl)-, —S—, —S(O)—, and —S(O)₂—, substituted with one substituent selected from the group consisting of —NH₂, —NHR¹¹, and —NR¹¹R¹², and further unsubstituted or substituted with one or two ═O substituents;

R⁷, R⁸, R⁹, R¹⁰, R¹¹, and R¹² are independently aryl, heteroaryl, heterocyclyl, or alkyl substituted with one or two substituents independently selected from the group consisting of aryl, heteroaryl, and heterocyclyl; and

X¹ is hydrogen or fluoride.

A second embodiment of this invention is directed to a process for making the compounds.

A third embodiment of this invention is directed to intermediates which are used in the second embodiment.

A fourth embodiment this invention is directed to compositions which are useful for the prophylaxis or treatment of penicillin-resistant streptococcus pneumoniae in a fish or a mammal, the compositions comprising a therapeutically effective amount of one or more of the compounds of the first embodiment and an excipient.

A fifth embodiment of this invention is directed to methods for prophylaxis or treatment of penicillin-resistant streptococcus pneumoniae in a fish or a mammal comprising administering to the fish or the mammal a therapeutically effective amount of one or more of the compounds of the first embodiment.

### DETAILED DESCRIPTION OF THE INVENTION

Compounds of this invention, also referred to as “the compounds,” comprise of both fixed and variable moieties, which variable moieties are identified by a capital letter and accompanying numerical or alphabetical superscript, and for which the following terms have the meanings indicated.

“Alkenyl” means monovalent, straight-chain and branched-chain hydrocarbon moieties, having two to eight carbon atoms and at least one carbon-carbon double bond. Alkenyl moieties include but-1,3-dienyl, butenyl, but-2-enyl, ethenyl, 1-ethylhexen-2-yl, hex-3-enyl, 1-methylbutenyl, 2-methylbutenyl, 1-methylbut-2-enyl, 1-methylbut-1,3-dienyl, pentenyl, pent-2-enyl, pent-3-enyl, and propenyl.

“Alkyl” means monovalent, saturated, straight-chain and branched-chain hydrocarbon moieties, having one to six carbon atoms. Alkyl moieties include butyl, 1,1,-dimethylethyl (tert-butyl), 1,1-dimethylpropyl, 1,2-dimethylpropyl, ethyl, 1-ethylpropyl, 2-ethylpropyl, hexyl, methyl, 2-methylpropyl, 3-methylbutyl, 1-methylpentyl, 2-methylpent-3-yl, and pentyl.

“Alkylene” means divalent, saturated, straight-chain and branched-chain hydrocarbon moieties, having one to eight carbon atoms. Alkylene moieties include butylene, 1,1,-dimethylethylene, 1,1-dimethylpropylene, 1,2-dimethylpropylene, ethylene, 1-ethylpropylene, 2-ethylpropylene, hexylene, methylene, 2-methylpropylene, 3-methylbutylene, 1-methylpentylene, 2-methylpent-3-ylene, and pentylene.

“Alkynyl” means monovalent, straight-chain and branched-chain hydrocarbon moieties, having two to six carbon atoms and at least one carbon-carbon triple bond. Alkynyl moieties include ethynyl (acetylenyl), pentynyl, pent-2-ynyl, pent-3-ynyl, pent-4-ynyl, 1-methylbut-2-ynyl, 2-methylbut-3-ynyl, hexynyl, hex-2-ynyl, hex-3-ynyl, hex-4-ynyl, 1-methyl-pent-2-ynyl, 1-methylenepent-3-ynyl, 1-methyl-pent-2,4-diynyl, and prop-2-ynyl (propargyl).

“Aryl” means monovalent, unsubstituted or substituted phenyl which is unfused or fused with another phenyl moiety or a cycloalkyl, cycloalkenyl, heteroaryl, heterocyclyl, naphthyl, or saturated part of an indanyl moiety.

Phenyl moieties fused with phenyl, naphthyl, or the saturated part of an indanyl moieties are unsubstituted and substituted naphthyl, anthracen-(1- to 4-)yl, or fluoren-(1- to 4-)yl, respectively.

Phenyl moieties fused with cycloalkyl moieties are unsubstituted and substituted indan-(4- to 7-)yl and 1,2,3,4-tetrahydronaphth-(5- to 8-)yl.

Phenyl moieties fused with cycloalkenyl moieties are unsubstituted and substituted inden-(4- to 7-)yl, 1,2-dihydronaphth-(5- to 8-)yl and 1,2-dihydronaphth-(5- to 8-)yl.

Phenyl moieties fused with heteroaryl moieties include unsubstituted and substituted benzimidazol-(4- to 7-)yl, 1-benzofuran-(4- to 7-)yl, 1,2-benzisothiazol-(4- to 7-)yl, benzthiazol-(4- to 7-)yl, 1-benzothiophen-(4- to 7-)yl, cinnolin-(5- to 8-)yl, indol-(4- to 7-)yl, isoquinolin-(5- to 8-)yl, phthalazin-(5- to 8-)yl, quinazolin-(5- to 8-)yl, quinolin-(5- to 8-)yl, and quinoxalin-(5- to 8-)yl.

Phenyl moieties fused with heterocyclyl moieties include unsubstituted and substituted 1,3-benzodioxa(4- to 7-)yl, 1,4-benzodioxa(5- to 8-)yl, 1,3-dihydro-2-benzofuran-(4- to 7-)yl, 2,3-dihydro-1-benzofuran-(4- to 7-)yl, 1,3-dihydro-2-benzothiophen-(4- to 7-)yl, 2,3-dihydro-1-benzothiophen-(4- to 7-)yl, and indolin-(4- to 7-)yl.

“Cycloalkyl” means monovalent, unsubstituted and substituted, saturated cyclic hydrocarbon moieties, having three to six carbon atoms. Cycloalkyl moieties are unsubstituted and substituted cyclopropyl, cyclobutyl, cyclopentyl, and cyclohexyl.

“Cycloalkenyl” means means monovalent, unsubstituted and substituted, cyclic hydrocarbon moieties having four to six carbon atoms and at least one carbon-carbon double bond. Cycloalkenyl moieties are unsubstituted and substituted 1,3-cyclohexadienyl, 1,4-cyclohexadienyl, cyclohexenyl, cyclopentadienyl, and cyclopentenyl.

“Halo” means fluoro (—F), chloro (—Cl), bromo (—Br), and iodo (—I).

“Heteroaryl” means monovalent, aromatic, unsubstituted and substituted five-membered ring moieties having two double bonds and (a) one oxygen or one sulfur atom, (b) one, two, three, or four nitrogen atoms, or (c) one or two nitrogen atoms and one oxygen or one sulfur atom and the remaining atoms are carbon atoms, each of which is attached through a carbon atom or a nitrogen atom; and monovalent six-membered ring moieties having three double bonds and one, two, or three nitrogen atoms and the remaining atoms are carbon atoms, attached through a carbon atom; in which the foregoing heteroaryl moieties are unfused or fused with another heteroaryl moiety or an aryl moiety.

Five-membered heteroaryl moieties are unsubstituted and substituted furanyl, imidazolyl, isothiazolyl, isoxazolyl, 1,2,3-oxadiazolyl, oxazolyl, pyrazolyl, pyrrolyl, tetrazolyl, 1,3,4-thiadiazolyl, thiazolyl, thiophenyl (thienyl), 2H-tetraäzolyl, and 1,2,3-triazolyl.

Five-membered heteroaryl moieties fused with aryl moieties include unsubstituted and substituted benzimidazol-(1- or 2-)yl, 1-benzofuran-(2- to 3-)yl, 1,2-benzisothiazol-3-yl, benzthiazol-2-yl, 1-benzothiophen-(2- to 3-)yl, cinnolin-(3- or 4-)yl, indol-(1- to 3-)yl, isoquinolin-(1-, 3-, or 4-)yl, phthalazin-(1- or 4-)yl, quinazolin-(2- or 4-)yl, quinolin-(2- to 4-)yl, and quinoxalin-(2- or 3-)yl.

Five-membered heteroaryl moieties fused with other five-membered heteroaryl moieties include unsubstituted and substituted [1,3]thiazolo[4,5-d][1,3]oxazolyl, [1,3]thiazolo[4,5-d][1,3]thiazolyl, thieno[3,2-d][1,3]oxazolyl, thieno[3,2-d][1,3]thiazolyl, and thieno[2,3-b]thiophenyl.

Five-membered heteroaryl moieties fused with six-membered heteroaryl moieties include unsubstituted and substituted furo[2,3-b]pyridin-(2- or 3-)yl, 3H-imidazo[4,5-b]pyridin-(2- or 3-)yl, [1,3]thiazolo[4,5-b]pyrazin-2-yl, [1,3]thiazolo[4,5-b]pyridin-2-yl, and thieno[2,3-b]pyridin-(2- or 3-)yl.

Six-membered heteroaryl moieties are unsubstituted and substituted pyrazinyl, pyridazinyl, pyridyl, pyrimidinyl, and 1,3,5-triazinyl.

Six-membered heteroaryl moieties fused with aryl moieties include unsubstituted and substituted cinnolin-(3- or 4-)yl, isoquinolin-(1-, 3-, or 4-)yl, phthalazin-(1- or 4-)yl, quinazolin-(2- or 4-)yl, quinolin-(2- to 4-)yl, and quinoxalin-(2- or 3-)yl.

Six-membered heteroaryl moieties fused with five-membered heteroaryl moieties include unsubstituted and substituted furo[2,3-b]pyridin-(4- to 6-)yl, 3H-imidazo[4,5-b]pyridin-(5- to 7-)yl, [1,3]thiazolo[4,5-b]pyrazin-(5- or 6-)yl, [1,3]thiazolo[4,5-b]pyridin-(5- to 7-)yl, and thieno[2,3-b]pyridin-(4- to 6-)yl.

Six-membered heteroaryl moieties fused with other six-membered heteroaryl moieties include unsubstituted and substituted 1,5-naphthyridinyl, 1,7-naphthyridinyl, 1,8-naphthyridinyl, pteridinyl, pyridazino[4,5-d]pyridazinyl, pyrido[2,3-d]pyridazinyl, and pyrido[3,4-d]pyridazinyl.

“Heterocyclyl” means (a) monovalent, non-aromatic, unsubstituted and substituted four-membered ring moieties having one nitrogen, oxygen, or sulfur atom and the remaining atoms are carbon atoms, zero double bonds, attached through a carbon atom or a nitrogen atom, (b) monovalent, non-aromatic, unsubstituted and substituted five-membered ring moieties having one or two nitrogen, oxygen, or sulfur atoms and the remaining atoms are carbon atoms, and zero or one double bonds, attached through a carbon atom or a nitrogen atom, and (c) monovalent, non-aromatic, unsubstituted and substituted six-membered ring moieties having one, two, or three nitrogen, oxygen, or sulfur atoms and the remaining atoms are carbon atoms, and zero, one, or two double bonds, attached through a carbon atom or a nitrogen atom.

Four-membered heterocyclyl moieties are unsubstituted and substituted oxetane, thietane, and azetidine.

Five-membered heterocyclyl moieties include unsubstituted and substituted 1,4-dioxanyl, 1,3-dioxolanyl, imidazolidinyl, 2-imidazolinyl, 4,5-dihydroisoxazolyl, pyrazolidinyl, 2-pyrazolinyl, pyrrolidinyl, 2-pyrrolinyl, 3-pyrrolinyl, and 2H-pyrrolyl.

Six-membered heterocyclyl moieties include unsubstituted and substituted 1,3-dithianyl, 1,4-dithianyl, morpholinyl, piperidinyl, piperazinyl, pyranyl, 2H-pyranyl, 4H-pyranyl, and thiomorpholinyl.

Substituted aryl and heteroaryl moieties are those moieties substituted with one, two, three, four, or five substituents independently selected from the group consisting of alkyl, alkenyl, alkynyl, cycloalkyl, halo, —CN, —OH, —SH, —NH₂, —NO₂, —CF₃, —CH₂CF₃, —CF₂CF₃, —OCF₃, —OCH₂CF₃, —OCF₂CF₃, —OR³⁰, —SR³⁰, —S(O)(alkyl), —SO₂(alkyl), —C(O)H, —C(O)(alkyl), —C(O)OH, —C(O)O(alkyl), —NH(alkyl), —N(alkyl)₂, —C(O)NH₂, —C(O)NH(alkyl), —C(O)N(alkyl)₂, —OC(O)(alkyl), —OC(O)O(alkyl), —OC(O)NH₂, —OC(O)NH(alkyl), —OC(O)N(alkyl)₂, —NHC(O)H, —NHC(O)(alkyl), —NHC(O)O(alkyl), —NHC(O)NH₂, —NHC(O)NH(alkyl), —NHC(O)N(alkyl)₂, —SO₂NH₂, —SO₂NH(alkyl), —SO₂N(alkyl)₂, and R⁴⁰, in which R³⁰ is alkyl or alkyl substituted with one substituent selected from the group consisting of halo, —O(alkyl), and —S(alkyl), and R⁴⁰ is furyl, imidazolyl, indazolidinyl, isoquinolyl, isothiazolyl, isoxazolyl, morpholinyl, naphthyl, naphthyridyl, 1,2,3-oxadiazolyl, oxazolyl, phenyl, piperidinyl, piperazinyl, pyrazinyl, pyrazolyl, pyridyl, pyrimidinyl, pyrrolidinyl, pyrrolyl, quinazolyl, quinolyl, quinoxalyl, tetrazolyl, 1,2,3-thiadiazolyl, 1,3,4-thiadiazolyl, thiazolyl, thienyl, 1,2,3-triazolyl, or thiomorpholinyl, in which each R⁴⁰ moiety is unsubstituted or substituted with one, two, or three substituents independently selected from the group consisting of alkyl, alkenyl, alkynyl, cycloalkyl, halo, ═O, —CN, —OH, —SH, —NO₂, —CF₃, —CH₂CF₃, —CF₂CF₃, —OCF₃, —OCH₂CF₃, —OCF₂CF₃, —O(alkyl), —S(alkyl), —S(O)(alkyl), —SO₂(alkyl), —C(O)H, —C(O)(alkyl), —C(O)OH, —C(O)O(alkyl), —NH₂, —NH(alkyl), —N(alkyl)₂, —C(O)NH₂, —C(O)NH(alkyl), —C(O)N(alkyl)₂, —OC(O)(alkyl), —OC(O)O(alkyl), —OC(O)NH₂, —OC(O)NH(alkyl), —OC(O)N(alkyl)₂, —NHC(O)H, —NHC(O)(alkyl), —NHC(O)O(alkyl), —NHC(O)NH₂, —NHC(O)NH(alkyl), —NHC(O)N(alkyl)₂, —SO₂NH₂, —SO₂NH(alkyl), and —SO₂N(alkyl)₂.

Substituted cycloalkyl, cycloalkenyl, and heterocyclyl moieties are those moieties substituted with one, two, or three substituents independently selected from the group consisting of alkyl, halo, —CN, —OH, —NH₂, —CF₃, —OR³⁰, —SR³⁰, —S(O)(alkyl), —SO₂(alkyl), —C(O)H, —C(O)(alkyl), —C(O)OH, —C(O)O(alkyl), —NH(alkyl), —N(alkyl)₂, —C(O)NH₂, —C(O)NH(alkyl), —C(O)N(alkyl)₂, and R⁴⁰, in which the phenyl is unsubstituted or substituted with one, two, or three substituents independently selected from the group consisting of halo, —CN, —OH, —NH₂, and —CF₃.

“Hydroxyl protecting moiety” means selectively introducible and removable moieties which protect —OH moieties against undesirable side reactions. Hydroxyl protecting moieties include 4-nitrobenzyloxycarbonyl, 4-bromobenzyloxycarbonyl, 4-methoxybenzyloxycarbonyl, 3,4-dimethoxybenzyloxycarbonyl, tert-butoxycarbonyl, diphenylmethoxycarbonyl, 2,2,2-trichloroethoxycarbonyl, 2,2,2-tribromoethoxycarbonyl, 2-(trimethylsilyl)ethoxycarbonyl, 2-(phenylsulfonyl)ethoxycarbonyl, allyloxycarbonyl, acetyl, chloroacetyl, dichloroacetyl, trichloroacetyl, trifluoroacetyl, methoxyacetyl, phenoxyacetyl, pivaloyl, propionyl, 2-methylpropionyl, benzoyl, tert-butyl, 2,2,2-trichloroethyl, 2-trimethylsilylethyl, 1,1-dimethyl-2-propenyl, 3-methyl-3-butenyl, para-methoxybenzyl, 3,4-dimethoxybenzyl, diphenylmethyl, triphenylmethyl, tetrahydrofuryl, benzyloxymethyl, 2-methoxyethoxymethyl, 2,2,2-trichloroethoxymethyl, 2-(trimethylsilyl)ethoxymethyl, methanesulfonyl, para-toluenesulfonyl, trimethylsilyl, triethylsilyl, triisopropylsilyl, diethylisopropylsilyl, tert-butyldimethylsilyl, tert-butyldiphenylsilyl, diphenylmethylsilyl, and tert-butylmethoxyphenylsilyl.

A specific example of R¹ and X¹ moieties for compounds having formula (I) is hydrogen.

Specific examples of R³ moieties for compounds having formula (I), are

(2-((3-(3-((3-(benzyloxy)benzyl)amino)propoxy)propyl)amino)ethyl)oxo)imino,

(2-((3-(3-(((9-ethyl-9H-carbazol-3-yl)methyl)amino)propoxy)propyl)amino)ethyl)oxo)imino,

((2-((3-(3-((3-(4-isopropylphenyl)-2-methylpropyl)amino)propoxy)proply)amino)ethyl)oxo)imino,

(2-((3-(3-((3-(4-methylphenoxy)benzyl)amino)propoxy)propyl)amino)ethyl)oxo)imino,

(((2-((3-(3-((4-phenylbenzyl)amino)propoxy)propyl)amino)ethyl)oxo)imino),

(((2-((3-(3-((3-phenoxybenzyl)amino)propoxy)propyl)amino)ethyl)oxo)imino),

(((2-((3-(3-((4-phenoxybenzyl)amino)propoxy)propyl)amino)ethyl)oxo)imino),

((2-((6-((3-phenoxybenzyl)amino)hexyl)amino)ethyl)oxo)imino, and

(((4-phenoxyphenyl)-6,9-dioxa-3,12-diazatridec-1-yl)oxo)imino.

These specific moieties of the compounds may combine with the fixed moieties thereof to form a sixth embodiment of this invention, which embodiment is directed to

compounds having formula (I), and salts, prodrugs, and salts of prodrugs thereof, in which R¹ is hydrogen; R³ is —CH₂R⁴; R⁴ is alkyl interrupted with two, three, or four moieties independently selected from the group consisting of —O— and —NH— and substituted with one —NHR⁷ substituent; R⁷ is alkyl substituted with one substituent selected from the group consisting of phenyl and phenyl fused with the pyrrolidine part of indoline; and X¹ is hydrogen, in which the R⁷ phenyl is substituted with one substituent selected from the group consisting of alkyl, phenyl, and —OR³⁰, and the pyrrolidine part of the R⁷ indoline is substituted with alkyl, in which R³⁰ is phenyl substituted with alkyl or alkyl substituted with phenyl;

compounds, and salts, prodrugs, and salts of prodrugs thereof, having formula (I) in which R¹ is hydrogen; R³ is —CH₂R⁴; R⁴ is C₈-alkyl interrupted with two, three, or four moieties independently selected from the group consisting of —O— and —NH— and substituted with one —NHR⁷ substituent; R⁷ is C₁-C₄-alkyl substituted with one substituent selected from the group consisting of phenyl and phenyl fused with the pyrrolidine part of indoline; and X¹ is hydrogen, in which the R⁷ phenyl is substituted with one substituent selected from the group consisting of C₃-alkyl, phenyl, and —OR³⁰, and the pyrrolidine part of the R⁷ indoline is substituted with C₂-alkyl, in which R³⁰ is phenyl substituted with C₁-alkyl or C₁-alkyl substituted with phenyl; and

compounds, and salts, prodrugs, and salts of prodrugs thereof, which include

(3R,5R,6R,7S,9R,10E,11S,12R,13S,14R)-6-(((2S,3R,4S,6R)-4-(dimethylamino)-3-hydroxy-6-methyltetrahydro-2H-pyran-2-yl)oxy)-14-ethyl-12,13-dihydroxy-7-methoxy-3,5,7,9,11,13-hexamethyloxacyclotetradecane-2,4,10-trione 10-(O-(2-((3-(3-((4-phenoxybenzyl)amino)propoxy)propyl)amino)ethyl)oxime);

(3R,5R,6R,7S,9R,10E,11S,12R,13S,14R)-6-(((2S,3R,4S,6R)-4-(dimethylamino)-3-hydroxy-6-methyltetrahydro-2H-pyran-2-yl)oxy)-14-ethyl-12,13-dihydroxy-7-methoxy-3,5,7,9,11,13-hexamethyloxacyclotetradecane-2,4,10-trione 10-(O-(2-((3-(3-((1,1′-biphenyl-4-ylmethyl)amino)propoxy)propyl)amino)ethyl)oxime);

(3R,5R,6R,7S,9R,10E,11S,12R,13S,14R)-6-(((2S,3R,4S,6R)-4-(dimethylamino)-3-hydroxy-6-methyltetrahydro-2H-pyran-2-yl)oxy)-14-ethyl-12,13-dihydroxy-7-methoxy-3,5,7,9,11,13-hexamethyloxacyclotetradecane-2,4,10-trione 10-(O-(2-((3-(3-((3-phenoxybenzyl)amino)propoxy)propyl)amino)ethyl)oxime);

(3R,5R,6R,7S,9R,10E,11S,12R,13S,14R)-6-(((2S,3R,4S,6R)-4-(dimethylamino)-3-hydroxy-6-methyltetrahydro-2H-pyran-2-yl)oxy)-14-ethyl-12,13-dihydroxy-7-methoxy-3,5,7,9,11,13-hexamethyloxacyclotetradecane-2,4,10-trione 10-(O-(2-((3-(3-((3-(4-methylphenoxy)benzyl)amino)propoxy)propyl)amino)ethyl)oxime);

(3R,5R,6R,7S,9R,10E,11S,12R,13S,14R)-6-(((2S,3R,4S,6R)-4-(dimethylamino)-3-hydroxy-6-methyltetrahydro-2H-pyran-2-yl)oxy)-14-ethyl-12,13-dihydroxy-7-methoxy-3,5,7,9,11,13-hexamethyloxacyclotetradecane-2,4,10-trione 10-(O-(2-((3-(3-((3-(benzyloxy)benzyl)amino)propoxy)propyl)amino)ethyl)oxime);

(3R,5R,6R,7S,9R,10E,11S,12R,13S,14R)-6-(((2S,3R,4S,6R)-4-(dimethylamino)-3-hydroxy-6-methyltetrahydro-2H-pyran-2-yl)oxy)-14-ethyl-12,13-dihydroxy-7-methoxy-3,5,7,9,11,13-hexamethyloxacyclotetradecane-2,4,10-trione 10-(O-(2-((3-(3-(((9-ethyl-9H-carbazol-3-yl)methyl)amino)propoxy)propyl)amino)ethyl)oxime);

(3R,5R,6R,7S,9R,10E,11S,12R,13S,14R)-6-(((2S,3R,4S,6R)-4-(dimethylamino)-3-hydroxy-6-methyltetrahydro-2H-pyran-2-yl)oxy)-14-ethyl-12,13-dihydroxy-7-methoxy-3,5,7,9,11,13-hexamethyloxacyclotetradecane-2,4,10-trione 10-(O-(2-((3-(3-((3-(4-isopropylphenyl)-2-methylpropyl)amino)propoxy)propyl)amino)ethyl)oxime);

(3R,5R,6R,7S,9R,10E,11S,12R,13S,14R)-6-(((2S,3R,4S,6R)-4-(dimethylamino)-3-hydroxy-6-methyltetrahydro-2H-pyran-2-yl)oxy)-14-ethyl-12,13-dihydroxy-7-methoxy-3,5,7,9,11,13-hexamethyloxacyclotetradecane-2,4,10-trione 10-(O-(2-((6-((3-phenoxybenzyl)amino)hexyl)amino)ethyl)oxime); and

(3R,5R,6R,7S,9R,10E,11S,12R,13S,14R)-6-(((2S,3R,4S,6R-4-(dimethylamino)-3-hydroxy-6-methyltetrahydro-2H-pyran-2-yl)oxy)-14-ethyl-12,13-dihydroxy-7-methoxy-3,5,7,9,11,13-hexamethyloxacyclotetradecane-2,4,10-trione 10-(O-(13-(4-phenoxyphenyl)-6,9-dioxa-3,12-diazatridec-1-yl)oxime).

Compounds of this invention contain asymmetrically substituted carbon atoms in the R or S configuration, in which the terms “R” and “S” are as defined by the IUPAC 1974 Recommendations for Section E, Fundamental Stereochemistry, Pure Appl. Chem. (1976) 45, 13-10. Compounds having asymmetrically substituted carbon atoms with equal amounts of R and S configurations are racemic at those carbon atoms. Atoms with an excess of one configuration over the other are assigned the configuration which is present in the higher amount, preferably an excess of about 85%-90%, more preferably an excess of about 95%-99%, and still more preferably an excess greater than about 99%. Accordingly, this invention is meant to embrace all stereoisomers of the compounds including racemic mixtures, enantiomers, mixtures of enantiomers, diastereomers, and mixtures of diastereomers.

Individual stereoisomers of the compounds may be prepared by any one of a number of methods within the knowledge of the ordinarily skilled practioner. These methods include stereospecific synthesis, chromatographic separation of diastereomers, chromatographic resolution of enantiomers, enzymatic resolution, and conversion of enantiomers in an enantiomeric mixture to diastereomers and chromatographically separating the diastereomers and regeneration of the individual enantiomers.

Stereospecific synthesis involves the use of appropriate chiral starting materials and synthetic reactions which do not cause racemization or inversion of stereochemistry at the chiral centers.

Diastereomeric mixtures of compounds resulting from a synthetic reaction can be separated by chromatographic techniques which are well-known to the ordinarily skilled practioner.

Chromatographic resolution of enantiomers can be accomplished on chiral commercially available chromatography resins. In practice, the racemate is placed in solution and loaded onto the column containing a chiral stationary phase. The enantiomers are then separated by high performance liquid chromatography.

Enzymes, such as esterases, phosphatases and lipases, may be useful for resolution of derivatives of the enantiomers in an enantiomeric mixture. For example, an ester derivative of a carboxyl group of the compounds to be separated can be prepared. Certain enzymes will selectively hydrolyze only one of the enantiomers in the mixture. Then the resulting enantiomerically pure acid can be separated from the unhydrolyzed ester.

Resolution of enantiomers may also be accomplished by converting the enantiomers in the mixture to diastereomers by reacting of the former and chiral auxiliaries. The resulting diastereomers can then be separated by column chromatography. This technique is especially useful when the compounds to be separated contain a carboxyl, amino or hydroxyl group that will form a salt or covalent bond with the chiral auxiliary. Chirally pure amino acids, organic carboxylic acids or organosulfonic acids are especially useful as chiral auxiliaries. Once the diastereomers have been separated by chromatography, the individual enantiomers can be regenerated. Frequently, the chiral auxiliary can be recovered and reused.

Compounds of this invention which contain hydroxyl, amino, or carboxylic acids may have attached thereto prodrug-forming moieties. The prodrug-forming moieties are removed by metabolic processes and release the compounds having the freed hydroxyl, amino, or carboxylic acid in vivo. Prodrugs are useful for adjusting such pharmacokinetic properties of the compounds as solubility and/or hydrophobicity, absorption in the gastrointestinal tract, bioavailability, tissue penetration, and rate of clearance.

Compounds of this invention may exist as acid addition salts, basic addition salts, or zwitterions. Salts of the compounds are prepared during their isolation or following their purification. Acid addition salts of the compounds are those derived from the reaction of the compounds with an acid. For example, the acetate, adipate, alginate, bicarbonate, citrate, aspartate, benzoate, benzenesulfonate, bisulfate, butyrate, camphorate, camphorsufonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetic, trifluoroacetic, para-toluenesulfonate, and undecanoate, salts of the compounds and prodrugs thereof are embraced by this invention. When the compounds contain carboxylic acids, basic addition salts may be prepared therefrom by reaction with a base such as the hydroxide, carbonate, and bicarbonate of cations such as lithium, sodium, potassium, calcium, and magnesium.

Compounds of this invention may be administered with or without an excipient. Excipients include encapsulating materials or formulation additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, and mixtures thereof. Excipients for orally administered compounds in solid dosage forms include agar, alginic acid, aluminum hydroxide, benzyl alcohol, benzyl benzoate, 1,3-butylene glycol, castor oil, cellulose, cellulose acetate, cocoa butter, corn starch, corn oil, cottonseed oil, ethanol, ethyl acetate, ethyl carbonate, ethyl cellulose, ethyl laureate, ethyl oleate, gelatin, germ oil, glucose, glycerol, groundnut oil, isopropanol, isotonic saline, lactose, magnesium hydroxide, magnesium stearate, malt, olive oil, peanut oil, potassium phosphate salts, potato starch, propylene glycol, Ringer's solution, talc, tragacanth, water, safflower oil, sesame oil, sodium carboxymethyl cellulose, sodium lauryl sulfate, sodiumphosphate salts, soybean oil, sucrose, tetrahydrofurfuryl alcohol, and mixtures. Excipients for ophthalmically and orally administered compounds in liquid dosage forms include benzyl alcohol, benzyl benzoate, 1,3-butylene glycol, castor oil, corn oil, cottonseed oil, ethanol, ethyl acetate, ethyl carbonate, fatty acid esters of sorbitan, germ oil, groundnut oil, glycerol, isopropanol, olive oil, polyethylene glycols, propylene glycol, sesame oil, tetrahydrofurfuryl alcohol, water, and mixtures thereof. Excipients for osmotically administered compounds include chlorofluorohydrocarbons, ethanol, isopropanol, water, and mixtures thereof. Excipients for parenterally administered compounds include 1,3-butanediol, castor oil, corn oil, cottonseed oil, germ oil, groundnut oil, liposomes, oleic acid, olive oil, peanut oil, Ringer's solution, safflower oil, sesame oil, soybean oil, U.S.P. or isotonic sodium chloride solution, water, and mixtures thereof. Excipients for rectally and vaginally administered compounds include cocoa butter, polyethylene glycol, wax, and mixtures thereof.

Compounds of this invention may be administered orally, ophthalmically, osmotically, parenterally (subcutaneously, intramuscularly, intrasternally, intravenously), rectally, topically, transdermally, and vaginally. Orally administered compounds in solid dosage forms may be administered as capsules, dragees, granules, pills, powders, and tablets. Ophthalmically and orally administered compounds in liquid dosage forms may be administered as elixirs, emulsions, microemulsions, solutions, suspensions, and syrups. Osmotically and topically administered compounds may be administered as creams, gels, inhalants, lotions, ointments, pastes, powders, solutions, and sprays. Parenterally administered compounds may be administered as aqueous or oleaginous solutions or aqueous or oleaginous and suspensions, in which suspensions comprise crystalline, amorphous, or otherwise insoluble forms of the compounds. Rectally and vaginally administered compounds may be administered as creams, gels, lotions, ointments, and pastes.

Therapeutically effective amounts of compounds of this invention depend on the recepient of treatment, the disorder being treated and the severity of the disorder, the composition comprising the compounds, the time of administration, the route of administration, the duration of treatment, the potency of the compounds, and the rate of excretion of the compounds. The daily therapeutically effective amount of the compounds administered to a patient in single or divided doses range from about 0.1 to about 200 mg/kg body weight, preferably from about 0.25 to about 100 mg/kg body weight. Single dose compositions contain these amounts of the compounds or combinations of submultiples thereof.

To determine antibacterial activity of compounds of this invention against penicillin-resistant streptococcus pneumoniae, twelve petri dishes, each containing successive aqueous dilutions of test compounds in sterilized Brain Heart Infusion agar (Difco 0418-01-5) (10 mL), were inoculated with 1:100 dilutions of penicillin-resistant streptococcus pneumoniae using a Steers replicator block, co-incubated at 35-37° C. for 20-24 hours with a control plate having no compound, and inspected visually to provide the minimum inhibitory concentration (MIC), in μg/mL, by which is meant the lowest concentration of the test compound which yielded no growth, a slight haze, or sparsely isolated colonies on the inoculums spot as compared to growth in the control plate.

Compounds of this invention displayed antibacterial activity against penicillin-resistant streptococcus pneumoniae superior to the control, which control demonstrated no antibacterial activity. This antibacterial activity demonstrates the usefulness of the compounds as antibacterials for the treatment or prophylaxis of penicillin-resistant streptococcus pneumoniae.

It is meant to be understood that certain metabolites of compounds of this invention, which metabolites are produced by in vitro or in vivo metabolic processes, would also be useful as antibacterials and are meant to be embraced by this invention.

It is also meant to be understood that certain precursor compounds, which precursor compounds may be metabolized in vitro or in vivo to form compounds of this invention, are meant to be embraced by this invention.

The compounds and processes of this invention will be better understood in connection with the following schemes and examples.

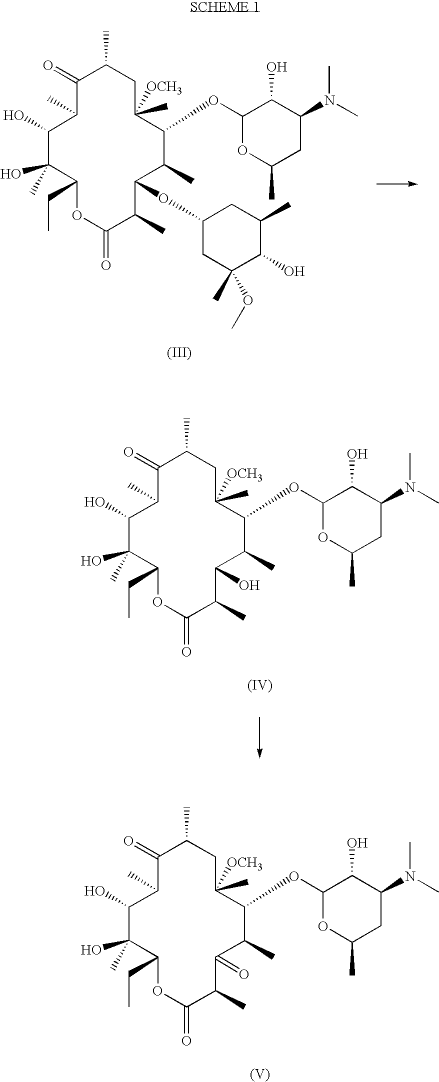

The compound having formula (III) (clarithromycin) may be converted to the compound having formula (IV) by reaction with dilute hydrochloric acid. The reaction is typically conducted in methanol, ethanol, isopropanol, butanol or mixtures thereof, over about 0.5 to about 24 hours, at about −10° C. to 70° C.

The compound having formula (IV) may be converted to the compound having formula (V) by reaction with N-chloro-succinimide.dimethyl sulfide or dicyclohexyl carbodiimide.dimethylsulfoxide and triethylamine or diisopropylethylamine in dichloromethane, over about 0.5 to about 4 hours at about −10° C. to 25° C.

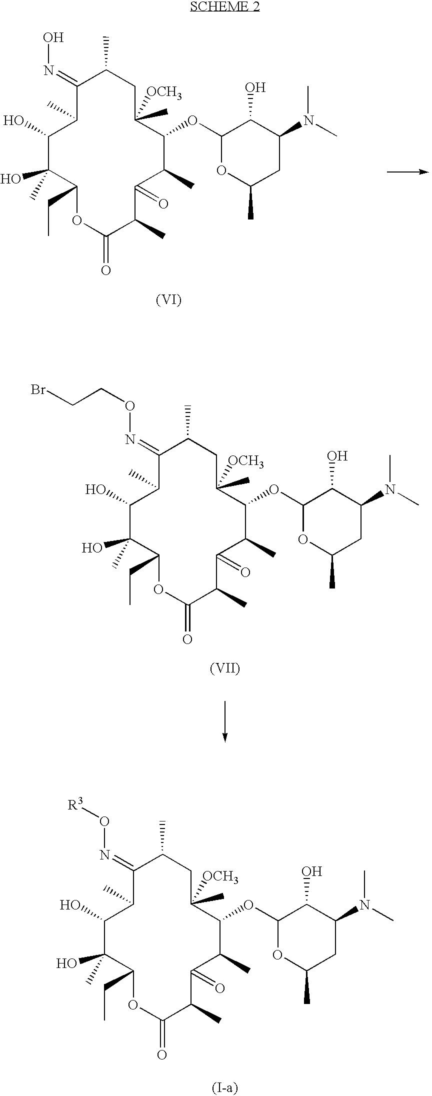

The compound having formula (V) may be converted to the compound having formula (VI) by reaction of the former and hydroxylamine hydrochloride and sodium acetate in ethanol, over about 1 to about 18 hours, at about 50° C. to about 80° C.

The compound having formula (VI) may be converted to the compound having formula (VII) by reaction of the former, 1,2-dibromoethane, and aqueous sodium hydroxide, over about 10 to about 18 hours, at about 35° C. to about 70° C.

The compound having formula (VII) may be converted to the compound having formula (I)-a by reaction of the former, the appropriate alkylating agent, and potassium carbonate, over about 10 to about 18 hours, at about 0° C. to about 30° C.

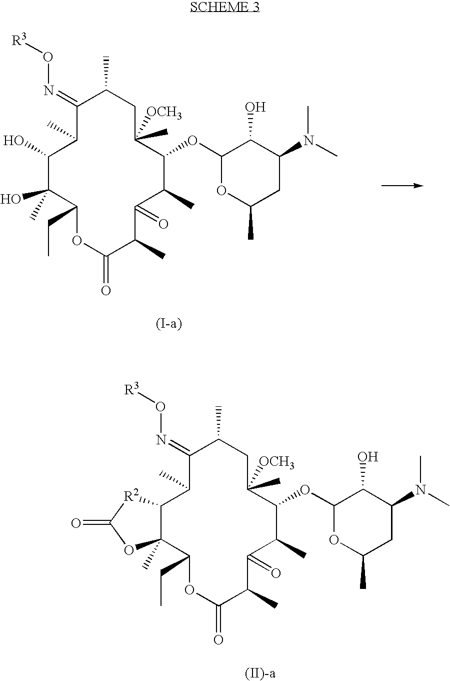

The compound having formula (I)-a may be converted to the compound having formula (II)-a as described in commonly-owned U.S. Pat. No. 5,866,549, the specification of which is hereby incorporated by reference into this application.

Compounds having formulas (I)-a and (II)-a may be fluoridated at the C-3 position of the macrolide as described in commonly-owned U.S. Pat. No. 6,124,269, the specification of which is hereby incorporated by reference into this application.

### EXAMPLE 1

### (3R,5R,6R,7S,9R,10E,11S,12R,13S,14R)-6-(((2S,3R,4S, 6R)-4-(dimethylamino)-3-hydroxy-6-methyltetrahydro-2H-pyran-2-yl)oxy)-14-ethyl-12,13-dihydroxy-7-methoxy-3,5,7,9,11,13-hexamethyloxacyclotetradecane-2,4,10-trione 10-(O-(2-bromoethyl)oxime)

A solution of the compound having formula (II) in SCHEME 1 (2.04 g), hydroxylamine hydrochloride (1.22 g), and sodium acetate (285 mg) in ethanol (35 mL) at 25° C. was stirred for 18 hours, heated at 75° C. for 18 hours and cooled, treated with dichloromethane, washed with water and saturated sodium bicarbonate, dried (Na₂SO₄), filtered, concentrated, and flash chromatographed on silica gel with 97:3:0.5 to 95:6:0.5 to 93:7:0.5 dichloromethane/methanol/concentrated ammonium hydroxide.

A mixture of the product from the preceeding paragraph (2.3 g), 1,2-dibromoethane (3.29 mL), 10% sodium hydroxide in water (38 mL), and tetrabutylammonium bromide (616 mg) at 40° C. was stirred for 8 hours and cooled, stirred for 18 hours, treated with dichloromethane, washed with water and brine, dried (Na₂SO₄), filtered, concentrated, and flash chromatographed on silica gel with 97:3:0.5 to 95:6:0.5 to 93:7:0.5 dichloromethane/methanol/concentrated ammonium hydroxide.

### EXAMPLE 2

A solution of EXAMPLE 1 (2.52 mmol), bis(3-aminopropyl)ether (5 mmol), and potassium carbonate (3.6 mmol) at 25° C. was stirred for 18 hours, filtered, concentrated, treated with ethyl acetate, washed with 10% sodium carbonate and brine, and dried (Na₂SO₄), filtered, and concentrated.

### EXAMPLE 3

A solution of 4-phenoxybenzaldehyde (0.06 mmol) in methanol (1 mL) at 25° C. was treated with EXAMPLE 2 (0.03 mmol) in methanol (0.5 mL), stirred for 1 hour, treated with DIEA (0.287 mmol), stirred for 2 hours, treated with borohydride resin (100 mg, 2.5 mmol/g), stirred for 4 hours, filtered, concentrated, and purified by reverse-phase column chromatography with an acetonitrile:water:gradient containing 0.1% trifluoroacetic acid.

### EXAMPLE 4

A solution of 4-phenylbenzaldehyde (0.06 mmol) in methanol (1 mL) at 25° C. was treated with EXAMPLE 2 (0.03 mmol) in methanol (0.5 mL), stirred for 1 hour, treated with DIEA (0.287 mmol), stirred for 2 hours, treated with borohydride resin (100 mg, 2.5 mmol/g), stirred for 4 hours, filtered, concentrated, and purified by reverse-phase column chromatography with acetonitrile:water:gradient containing 0.1% trifluoroacetic acid.

### EXAMPLE 5

A solution of 3-phenoxybenzaldehyde (0.06 mmol) in methanol (1 mL) at 25° C. was treated with EXAMPLE 2 (0.03 mmol) in methanol (0.5 mL), stirred for 1 hour, treated with DIEA (0.287 mmol), stirred for 2 hours, treated with borohydride resin (100 mg, 2.5 mmol/g), stirred for 4 hours, filtered, concentrated, and purified by reverse-phase column chromatography with an acetonitrile:water:gradient containing 0.1% trifluoroacetic acid.

### EXAMPLE 6

A solution of 3-(4-methylphenoxy)benzaldehyde (0.06 mmol) in methanol (1 mL) at 25° C. was treated with EXAMPLE 2 (0.03 mmol) in methanol (0.5 mL), stirred for 1 hour, treated with DIEA (0.287 mmol), stirred for 2 hours, treated with of borohydride resin (100 mg, 2.5 mmol/g), stirred for 4 hours, filtered, concentrated, and purified by reverse-phase column chromatography with an acetonitrile:water:gradient containing 0.1% trifluoroacetic acid.

### EXAMPLE 7

A solution of 3-benzyloxybenzaldehyde (0.06 mmol) in methanol (1 mL) at 25° C. was treated with EXAMPLE 2 (0.03 mmol) in methanol (0.5 mL), stirred for 1 hour, treated with DIEA (0.287 mmol), stirred for 2 hours, treated with borohydride resin (100 mg, 2.5 mmol/g), stirred for 4 hours, filtered, concentrated, and purified by reverse-phase column chromatography with an acetonitrile:water:gradient containing 0.1% trifluoroacetic acid.

### EXAMPLE 8

A solution of 9-ethyl-3-carbazolecarboxaldehyde (0.06 mmol) in methanol (1 mL) at 25° C. was treated with EXAMPLE 2 (0.03 mmol) in methanol (0.5 mL), stirred for 1 hour, treated with DIEA (0.287 mmol), stirred for 2 hours, treated with of borohydride resin (100 mg, 2.5 mmol/g), stirred for 4 hours, filtered, concentrated, and purified by reverse-phase column chromatography with an acetonitrile:water:gradient containing 0.1% trifluoroacetic acid.

### EXAMPLE 9

A solution of 3-(4-isopropylphenyl)-2-methylpropionaldehyde (0.06 mmol) in methanol (1 mL) at 25° C. was treated with EXAMPLE 2 (0.03 mmol) in methanol (0.5 mL), stirred for 1 hour, treated with DIEA (0.287 mmol), stirred for 2 hours, treated with of borohydride resin (100 mg, 2.5 mmol/g), stirred for 4 hours, filtered, concentrated, and purified by reverse-phase column chromatography with an acetonitrile:water:gradient containing 0.1% trifluoroacetic acid.

### EXAMPLE 10

A solution of EXAMPLE 1 (2.52 mmol), hexamethylenediamine (5 mmol), and potassium carbonate (3.6 mmol) at 25° C. was stirred for 18 hours, filtered, concentrated, treated with ethyl acetate, washed with 10% sodium carbonate and brine, and dried (Na₂SO₄), filtered, and concentrated.

### EXAMPLE 11

A solution of 3-phenoxybenzaldehyde (0.06 mmol) in methanol (1 mL) at 25° C. was treated with EXAMPLE 10 (0.03 mmol) in methanol (0.5 mL), stirred for 1 hour, treated with DIEA (0.287 mmol), stirred for 2 hours, treated with borohydride resin (100 mg, 2.5 mmol/g), stirred for 4 hours, filtered, concentrated, and purified by reverse-phase column chromatography with an acetonitrile:water:gradient containing 0.1% trifluoroacetic acid.

### EXAMPLE 12

A solution of EXAMPLE 1 (2.52 mmol), 2,2′-(ethylenedioxy)bis(ethylamine) (5 mmol), and potassium carbonate (3.6 mmol) at 25° C. was stirred for 18 hours, filtered, concentrated, treated with ethyl acetate, washed with 10% sodium carbonate and brine, and dried (Na₂SO₄), filtered, and concentrated.

### EXAMPLE 13

A solution of 4-phenoxybenzaldehyde (0.06 mmol) in methanol (1 mL) at 25° C. was treated with EXAMPLE 12 (0.03 mmol) in methanol (0.5 mL), stirred for 1 hour, treated with DIEA (0.287 mmol), stirred for 2 hours, treated with borohydride resin (100 mg, 2.5 mmol/g), stirred for 4 hours, filtered, concentrated, and purified by reverse-phase column chromatography with an acetonitrile:water:gradient containing 0.1% trifluoroacetic acid.

### SPECTRAL DATA

### EXAMPLE 3

¹H NMR (500 MHz, CD₃OD) δ7.47 (m, 2H), 7.39 (m, 2H), 7.17 (m, 1H), 7.03 (m, 4H), 5.22 (dd, J=11.0, 2.2 Hz, 1H), 4.41 (d, J=6.2 Hz, 1H), 4.36 (d, J=6.6 Hz, 1H), 4.27 (m, 2H), 4.19 (s, 2H), 4.02 (m, 1H), 3.88 (s, 1H), 3.74 (m, 1H), 3.66 (m, 1H), 3.57 (m, 4H), 3.43 (m, 3H), 3.21 (m, 2H), 3.14 (m, 6H), 2.87 (s, 3H), 2.79 (s, 3H), 2.77 (s, 3H), 2.72 (m, 2H), 1.98 (m, 7H), 1.55 (m, 5H), 1.40 (s, 3H), 1.33 (d, J=6.2 Hz, 3H), 1.31 (d, J=7.7 Hz, 3H), 1.30 (s, 3H), 1.26 (d, J=6.6 Hz, 3H), 1.19 (d, J=7.3 Hz, 3H), 1.01 (d, J=7 Hz, 3H), 0.87 (t, J=7.5 Hz, 3H).

### EXAMPLE 4

¹H NMR (500 MHz, CD₃OD) δ7.73 (m, 2H), 7.63 (m, 2H), 7.56 (m, 2H), 7.46 (m, 2H), 7.37 (m, 1H), 5.23 (dd, J=10.9, 2.5 Hz, 1H), 4.40 (d, J=6.2 Hz, 1H), 4.36 (d, J=6.9 Hz, 1H), 4.26 (m, 4H), 4.03 (m, 1H), 3.87 (m, 1H), 3.73 (m, 2H), 3.57 (m, 4H), 3.42 (m, 3H), 3.16 (m, 6H), 2.87 (s, 3H), 2.79 (s, 3H), 2.76 (s, 3H), 2.65 (s, 3H), 2.00 (m, 6H), 1.53 (m, 4H), 1.39 (s, 3H), 1.33 (d, J=5.9 Hz, 3H), 1.31 (d, J=9 Hz, 3H), 1.30 (s, 3H), 1.26 (d, J=6.6 Hz, 3H), 1.18 (d, J=7.2 Hz, 3H), 1.00 (d, J=6.9 Hz, 3H), 0.86 (t, J=7.5 Hz, 3H).

### EXAMPLE 5

¹H NMR (500 MHz, CD₃OD) δ7.45 (m, 1H), 7.38 (m, 2H), 7.19 (m, 3H), 7.03 (m, 3H), 5.22 (dd, J=10.9, 2.5 Hz, 1H), 4.40 (d, J=6.6 Hz, 1H), 4.36 (d, J=6.9 Hz, 1H), 4.27 (m, 2H), 4.20 (s, 2H), 4.01 (m, 1H), 3.87 (s, 1H), 3.69 (m, 2H), 3.56 (m, 4H), 3.43 (m, 3H), 3.17 (m, 6H), 2.87 (s, 3H), 2.79 (s, 3H), 2.76 (s, 3H), 2.73 (s, 1H), 2.65 (s, 3H), 1.87-2.09 (m, 7H), 1.55 (m, 5H), 1.40 (s, 3H), 1.33 (d, J=6.2 Hz, 3H), 1.31 (d, J=8.1 Hz, 3H), 1.30 (s, 3H), 1.25 (d, J=6.9 Hz, 3H), 1.19 (d, J=7.2 Hz, 3H), 1.01 (d, J=7.2 Hz, 3H), 0.86 (t, J=7.3 Hz, 3H).

### EXAMPLE 6

¹H NMR (500 MHz, CD₃OD) δ7.42 (m, 1H), 7.19 (m, 3H), 7.11 (m, 1H), 7.02 (m, 1H), 6.91 (m, 2H), 5.22 (dd, J=10.9, 2.5 Hz, 1H), 4.40 (d, J=6.2 Hz, 1H), 4.36 (d, J=6.9 Hz, 1H), 4.27 (m, 2H), 4.18 (s, 3H), 4.02 (m, 1H), 3.87 (s, 1H), 3.69 (m, 2H), 3.55 (m, 4H), 3.42 (m, 3H), 3.17 (m, 6H), 2.87 (s, 3H), 2.79 (s, 3H), 2.76 (s, 3H), 2.65 (s, 3H), 2.33 (s, 3H), 1.86-2.07 (m, 6H), 1.55 (m, 4H), 1.40 (s, 3H), 1.33 (d, J=6.2 Hz, 3H), 1.31 (d, J=8.1 Hz, 3H), 1.30 (s, 3H), 1.25 (d, J=6.9 Hz, 3H), 1.19 (d, J=6.9 Hz, 3H), 1.01 (d, J=6.9 Hz, 3H), 0.89 (m, 1H), 0.86 (t, J=7.3 Hz, 3H.

### EXAMPLE 7

¹H NMR (500 MHz, CD₃OD) δ7.39 (m, 6H), 7.10 (m, 3H), 5.22 (dd, J=10.9, 2.5 Hz, 1H), 5.12 (s, 2H), 4.40 (d, J=6.2 Hz, 1H), 4.36 (d, J=6.9 Hz, 1H), 4.26 (m, 2H), 4.18 (s, 2H), 4.02 (m, 1H), 3.87 (s, 1H), 3.70 (m, 2H), 3.56 (m, 4H), 3.43 (m, 3H), 3.17 (m, 6H), 2.87 (s, 3H), 2.79 (s, 3H), 2.76 (s, 3H), 2.65 (s, 3H), 1.98 (m, 6H), 1.55 (m, 5H), 1.40 (s, 3H), 1.33 (d, J=5.9 Hz, 3H), 1.31 (d, J=8.4 Hz, 3H), 1.30 (s, 3H), 1.26 (d, J=6.9 Hz, 3H), 1.18 (d, J=7.2 Hz, 3H), 1.01 (d, J=6.9 Hz, 3H), 0.86 (t, J=7.5 Hz, 3H).

### EXAMPLE 8

¹H NMR (500 MHz, CD₃OD) δ8.24 (m, 1H), 8.12 (d, J=7.8 Hz, 1H), 7.56 (m, 4H), 7.25 (m, 1H), 5.22 (dd, J=10.9, 2.5 Hz, 1H), 4.47 (q, J=7.2 Hz, 2H), 4.39 (m, 5H), 4.30 (m, 1H), 4.18 (m, 1H), 4.02 (m, 1H), 3.86 (m, 1H), 3.69 (m, 1H), 3.57 (m, 5H), 3.43 (m, 3H), 3.19 (m, 6H), 3.08 (m, 2H), 2.87 (s, 3H), 2.78 (s, 3H), 2.74 (s, 3H), 2.65 (s, 32H), 1.98 (m, 6H), 1.54 (m, 4H), 1.40 (m, 6H), 1.29 (m, 12H), 1.17 (m, 3H), 1.00 (m, 3H), 0.87 (m, 3H).

### EXAMPLE 9

¹H NMR (500 MHz, CD₃OD) δ7.18 (d, J=8.1 Hz, 2H), 7.11 (d, J=8.1 Hz, 2H), 5.22 (dd, J=10.8, 2.3 Hz, 1H), 4.41 (d, J=6.2 Hz, 1H), 4.36 (d, J=6.9 Hz, 1H), 4.26 (m, 4H), 4.02 (m, 1H), 3.87 (m, 1H), 3.69 (m, 2H), 3.56 (m, 4H), 3.44 (m, 3H), 3.04-3.25 (m, 8H), 2.99 (m, 1H), 2.87 (s, 3H), 2.79 (s, 3H), 2.76 (s, 3H), 2.65 (s, 3H), 2.49 (m, 1H), 2.14 (m, 1H), 1.98 (m, 9H), 1.55 (m, 6H), 1.40 (s, 3H), 1.33 (d, J=4.1 Hz, 3H), 1.32 (d, J=3.4 Hz, 3H), 1.30 (s, 3H), 1.26 (d, J=6.9 Hz, 3H), 1.23 (d, J=6.9 Hz, 6H), 1.19 (d, J=6.9 Hz, 3H), 1.00 (d, J=5.6 Hz, 3H), 0.87 (t, J=7.3 Hz, 3H).

### EXAMPLE 11

¹H NMR (500 MHz, CD₃OD) δ7.44 (m, 1H), 7.38 (m, 2H), 7.18 (m, 3H), 7.06 (m, 1H), 7.01 (m, 2H), 5.23 (dd, J=10.9, 2.5 Hz, 1H), 4.41 (d, J=6.2 Hz, 1H), 4.36 (d, J=6.9 Hz, 1H), 4.27 (m, 3H), 4.17 (s, 2H), 4.02 (m, 1H), 3.86 (s, 1H), 3.74 (m, 1H), 3.67 (m, 1H), 3.43 (m, 4H), 3.19 (m, 2H), 3.04 (m, 4H), 2.84 (m, 6H), 2.77 (s, 3H), 2.65 (s, 3H), 2.04 (m, 2H), 1.93 (m, 1H), 1.74 (m, 4H), 1.55 (m, 4H), 1.45 (m, 4H), 1.40 (s, 3H), 1.34 (d, J=5.9 Hz, 3H), 1.31 (s, 3H), 1.31 (d, J=7.2 Hz, 3H), 1.26 (d, J=6.6 Hz, 3H), 1.19 (d, J=7.2 Hz, 3H), 1.01 (d, J=6.9 Hz, 3H), 0.87 (t, J=7.3 Hz, 3H).

### EXAMPLE 13

¹H NMR (500 MHz, CD₃OD) δ7.48 (m, 2H), 7.38 (m, 2H), 7.17 (m, 1H), 7.03 (m, 4H), 5.23 (dd, J=10.9, 2.5 Hz, 1H), 4.40 (d, J=6.2 Hz, 1H), 4.36 (d, J=6.9 Hz, 1H), 4.29 (m, 2H), 4.22 (s, 2H), 4.02 (m, 1H), 3.82 (s, 1H), 3.78 (m, 2H), 3.72 (m, 4H), 3.65 (m, 2H), 3.41 (m, 5H), 3.22 (m, 3H), 2.85 (m, 2H), 2.76 (s, 3H), 2.76 (m, 6H), 2.71 (m, 2H), 2.04 (m, 2H), 1.92 (m, 2H), 1.54 (m, 7H), 1.40 (s, 3H), 1.33 (d, J=5.9 Hz, 3H), 1.31 (s, 3H), 1.31 (d, J=5.9 Hz, 3H), 1.26 (d, J=6.9 Hz, 3H), 1.18 (d, J=7.2 Hz, 3H), 1.00 (d, J=6.9 Hz, 3H), 0.86 (t, J=7.3 Hz, 3H).

The foregoing is merely illustrative of the invention and is not intended to limit the same to the disclosed compounds and processes. Variations and changes which are obvious to one skilled in the art are intended to be within the scope and nature of the invention as defined in the claims.

What is claimed is:

1. A compound, or a salt, prodrug, or a salt of a prodrug thereof, having formula (I) 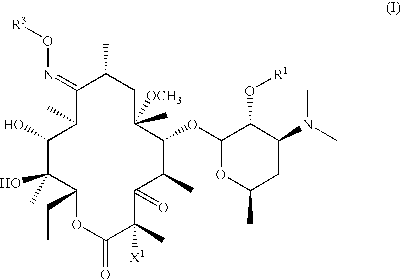
or formula (II) 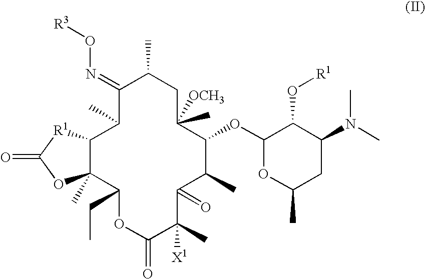
in which
R¹ is hydrogen or R^{P}, in which R^{P} is a hydroxyl protecting moiety;
R² is —O— or —NH—;
R³ is —CH₂R⁴, —CH₂CH₂R⁵, or —CH₂CH₂R⁶;
R⁴ is alkyl interrupted with one, two, three, or four moieties independently selected from the group consisting of —O—, ═N—, —NH—, —N(alkyl)-, —S—, —S(O)—, and —S(O)₂—, substituted with one substituent selected from the group consisting of —NH₂, —NHR⁷, and —NR⁷R⁸, and further unsubstituted or substituted with one or two ═O substituents;
R⁵ is alkenyl interrupted with one or two moieties independently selected from the group consisting of —O—, ═N—, —NH—, —N(alkyl)-, —S—, —S(O)—, and —S(O)₂—, substituted with one substituent selected from the group consisting of —NH₂, —NHR⁹, and —NR⁹R¹⁰, and further unsubstituted or substituted with one or two ═O substituents;
R⁶ is alkynyl interrupted with one or two moieties independently selected from the group consisting of —O—, ═N—, —NH—, —N(alkyl)-, —S—, —S(O)—, and —S(O)₂—, substituted with one substituent selected from the group consisting of —NH₂, —NHR¹¹, and —NR¹¹R¹², and further unsubstituted or substituted with one or two ═O substituents;
R⁷, R⁸, R⁹, R¹⁰, R¹¹, and R¹² are independently aryl, heteroaryl, heterocyclyl, or alkyl substituted with one or two substituents independently selected from the group consisting of aryl, heteroaryl, and heterocyclyl; and
X¹ is hydrogen or fluoride.

2. A compound of claim 1, or a salt, prodrug, or a salt of a prodrug thereof, having formula (I) 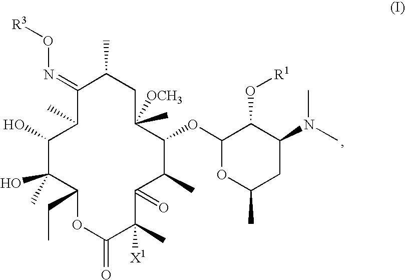
in which R¹ is hydrogen; R³ is —CH₂R⁴; R⁴ is alkyl interrupted with two, three, or four moieties independently selected from the group consisting of —O— and —NH— and substituted with one —NHR⁷ substituent; R⁷ is alkyl substituted with one substituent selected from the group consisting of phenyl and phenyl fused with the pyrrolidine part of indoline; and X¹ is hydrogen,
in which the R⁷ phenyl is substituted with one substituent selected from the group consisting of alkyl, phenyl, and —OR³⁰, and the pyrrolidine part of the R⁷ indoline is substituted with alkyl,
in which R³⁰ is phenyl or alkyl substituted with phenyl,
in which the R³⁰ phenyl is substituted with alkyl.

3. A compound of claim 1, or a salt, prodrug, or a salt of a prodrug thereof, having formula (I) 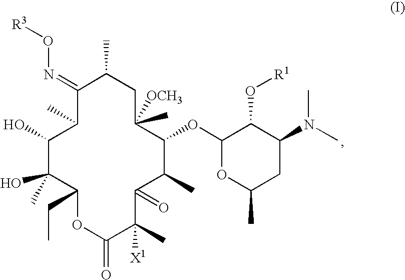
in which R¹ is hydrogen; R³ is —CH₂R⁴; R⁴ is C₈-alkyl interrupted with two, three, or four moieties independently selected from the group consisting of —O— and —NH— and substituted with one —NHR⁷ substituent; R⁷ is C₁-C₄-alkyl substituted with one substituent selected from the group consisting of phenyl and phenyl fused with the pyrrolidine part of indoline; and X¹ is hydrogen,
in which the R⁷ phenyl is substituted with one substituent selected from the group consisting of C₃-alkyl, phenyl, and —OR³⁰, and the pyrrolidine part of the R⁷ indoline is substituted with C₂-alkyl,
in which R³⁰ is phenyl or C₁-alkyl substituted with phenyl,
in which the R³⁰ phenyl is substituted with C₁-alkyl.

4. A composition for prophylaxis or treatment of penicillin-resistant streptococcus pneumoniae in a fish or a mammal comprising a therapeutically effective amount of a compound of claim 1 and an excipient.

5. A method for prophylaxis or treatment of penicillin-resistant streptococcus pneumoniae in a fish or a mammal comprising administering to the fish of the mammal a therapeutically effective amount of a compound of claim 1.

6. A compound, or a therapeutically acceptable salt, prodrug, or salt of a prodrug thereof, which is
(3R,5R,6R,7S,9R,10E,11S,12R,13S,14R)-6-(((2S,3R,4S,6R)-4-(dimethylamino)-3-hydroxy-6-methyltetrahydro-2H-pyran-2-yl)oxy)-14-ethyl-12,13-dihydroxy-7-methoxy-3,5,7,9,11,13-hexamethyloxacyclotetradecane-2,4,10-trione 10-(O-(2-((3-(3-((4-phenoxybenzyl)amino)propoxy)propyl)amino)ethyl)oxime);
(3R,5R,6R,7S,9R,10E,11S,12R,13S,14R)-6-(((2S,3R,4S,6R)-4-(dimethylamino)-3-hydroxy-6-methyltetrahydro-2H-pyran-2-yl)oxy)-14-ethyl-12,13-dihydroxy-7-methoxy-3,5,7,9,11,13-hexamethyloxacyclotetradecane-2,4,10-trione 10-(O-(2-((3-(3-((1,1′-biphenyl-4-ylmethyl)amino)propoxy)propyl)amino)ethyl)oxime);
(3R,5R,6R,7S,9R,10E,11S,12R,13S,14R)-6-(((2S,3R,4S,6R)-4-(dimethylamino)-3-hydroxy-6-methyltetrahydro-2H-pyran-2-yl)oxy)-14-ethyl-12,13-dihydroxy-7-methoxy-3,5,7,9,11,13-hexamethyloxacyclotetradecane-2,4,10-trione 10-(O-(2-((3-(3-((3-phenoxybenzyl)amino)propoxy)propyl)amino)ethyl)oxime);
(3R,5R,6R,7S,9R,10E,11S,12R,13S,14R)-6-(((2S,3R,4S,6R)-4-(dimethylamino)-3-hydroxy-6-methyltetrahydro-2H-pyran-2-yl)oxy)-14-ethyl-12,13-dihydroxy-7-methoxy-3,5,7,9,11,13-hexamethyloxacyclotetradecane-2,4,10-trione 10-(O-(2-((3-(3-((3-(4-methylphenoxy)benzyl)amino)propoxy)propyl)amino)ethyl)oxime;
(3R,5R,6R,7S,9R,10E,11S,12R,13S,14R)-6-(((2S,3R,4S,6R)-4-(dimethylamino)-3-hydroxy-6-methyltetrahydro-2H-pyran-2-yl)oxy)-14-ethyl-12,13-dihydroxy-7-methoxy-3,5,7,9,11,13-hexamethyloxacyclotetradecane-2,4,10-trione 10-(O-(2-((3-(3-((3-(benzyloxy)benzyl)amino)propoxy)propyl)amino)ethyl)oxime);
(3R,5R,6R,7S,9R,10E,11S,12R,13S,14R)-6-(((2S,3R,4S,6R)-4-(dimethylamino)-3-hydroxy-6-methyltetrahydro-2H-pyran-2-yl)oxy)-14-ethyl-12,13-dihydroxy-7-methoxy-3,5,7,9,11,13-hexamethyloxacyclotetradecane-2,4,10-trione 10-(O-(2-((3-(3-(((9-ethyl-9H-carbazol-3-yl)methyl)amino)propoxy)propyl)amino)ethyl)oxime);
(3R,5R,6R,7S,9R,10E,11S,12R,13S,14R)-6-(((2S,3R,4S,6R)-4-(dimethylamino)-3-hydroxy-6-methyltetrahydro-2H-pyran-2-yl)oxy)-14-ethyl-12,13-dihydroxy-7-methoxy-3,5,7,9,11,13-hexamethyloxacyclotetradecane-2,4,10-trione 10-(O-(2-((3-(3-((3-(4-isopropylphenyl)-2-methylpropyl)amino)propoxy)propyl)amino)ethyl)oxime;
(3R,5R,6R,7S,9R,10E,11S,12R,13S,14R)-6-(((2S,3R,4S,6R)-4-(dimethylamino)-3-hydroxy-6-methyltetrahydro-2H-pyran-2-yl)oxy)-14-ethyl-12,13-dihydroxy-7-methoxy-3,5,7,9,11,13-hexamethyloxacyclotetradecane-2,4,10-trione 10-(O-(2-((6-((3-phenoxybenzyl)amino)hexyl)amino)ethyl)oxime); or
(3R,5R,6R,7S,9R,10E,11S,12R,13S,14R)-6-(((2S,3R,4S,6R)-4-(dimethylamino)-3-hydroxy-6-methyltetrahydro-2H-pyran-2-yl)oxy)-14-ethyl-12,13-dihydroxy-7-methoxy-3,5,7,9,11,13-hexamethyloxacyclotetradecane-2,4,10-trione 10-(O-(13-(4-phenoxyphenyl)-6,9-dioxa-3,12-diazatridec-1-yl)oxime).
